(19) United States
(12) Patent Application Publication
(10) Pub. No.: US 2010/0035798 A1
(43) Pub. Date: Feb. 11, 2010

(54) METHODS AND COMPOSITIONS FOR THE TOPICAL OXYGENATION OF HYPOXIC TISSUE

(75) Inventors: Chandan K. Sen, New Albany, OH (US); Sashwati Roy, New Albany, OH (US); Savita Khanna, New Albany, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 12/441,523

(22) PCT Filed: Sep. 17, 2007
(86) PCT No.: PCT/US2007/078674

§ 371 Date: Mar. 16, 2009

Related U.S. Application Data
(60) Provisional application No. 60/825,848, filed on Sep. 15, 2006.

(51) Int. Cl. A61K 38/16; A61P 17/02; A61M 37/00

(52) U.S. Cl. 514/6; 600/21

(57) ABSTRACT

The invention provides methods, delivery devices, and compositions for topically oxygenating hypoxic tissue with a hemoglobin-based oxygen earner (HBOC), such as Oxyglobin (Oxyb) in order to promote wound healing and/or reducing hypoxia in post-harvest transplant tissue. The invention can be used, among other things, to stimulate angiogenesis and blood flow at a wound site, reduce wound dilation, and promote wound contraction, thereby increasing the overall rate of wound healing. The invention can also be used, among other things, to reduce oxidative stress in pre-implantation donor organ tissue, as well as stimulate the proliferation of granulation tissue, vessel growth, and promote epithelial healing in transplant tissue once it has been implanted.

### PRIORITY

This application claims priority of U.S. Application No. 60/825,848, filed Sep. 15, 2006, the disclosure of which is incorporated herein in its entirety.

### DESCRIPTION OF THE INVENTION

1. Field of the Invention

The invention relates generally to methods and compositions for the topical oxygenation of hypoxic tissues, such as wound tissue and harvested organs.

2. Background of the Invention

Hypoxia is a common characteristic of chronic wounds and results from, among other things, disrupted vasculature, peripheral vasculopathies, pulmonary disorders, and/or energy demands of regenerating tissue. Acutely, hypoxia promotes cell survival by expediting oxygen delivery to the oxygen-deprived tissues by increasing glucose transport, by raising the levels of glycolytic enzymes, and by inducing the expression of angiogenic factors. However, chronic hypoxia limits proliferation of dermal cells and causes tissue death and dysfunction. Because correction of wound hypoxia accelerates the proliferation of granulation tissue, stimulates vessel growth, and promotes epithelial healing, it is believed that tissue oxygenation is a key determinant of wound healing.

Clinical evidence suggests systemic oxygenation by hyperbaric oxygen therapy (HBOT), which increases the dissolved fraction of oxygen in plasma, promotes wound healing. However, one of the problems with HBOT is the lack of efficient means of oxygenating wound tissue while maintaining a low risk of systemic toxicity. Because superoxygenation (pO₂ multi-fold higher than basal pO₂ of the corresponding healthy tissue) may cause oxygen insult and stall regeneration, HBOT poses significant risk of oxygen toxicity to vital organs such as the brain and lungs. In the clinic, HBOT “dose” (time and pressure) is not optimized based on the need to correct wound hypoxia. For some patients the arbitrary “dose” creates oxygen toxicity and does not have a beneficial effect on wound healing. Also, because systemic delivery of oxygen to dermal wound tissue via HBOT relies on vascular sufficiency at the wound site, ischemic wounds and wounds otherwise complicated by vascular insufficiency (such as with diabetes) are not good candidates for receiving oxygen delivered systemically. Additionally, HBOT is cost intensive, available only at limited facilities, and is not a feasible means of treatment in a home or field setting.

In addition to HBOT, another means of systemically oxygenating tissues—blood substitutes that mimic the oxygen transporting ability of blood—has been developed for use in transfusions. One category of such substitutes is that of hemoglobin-based oxygen carriers (HBOCs), which are hemoglobin derivatives of human, animal, and/or artificial (i.e. via recombinant technology) origin. Although HBOCs have received much attention, a number of shortcomings exist with respect to their systemic use, namely that HBOCs are viewed as foreign substances in the body and thus the reticuloendothelial system acts to quickly clear them (the plasma half-life is generally 24 hours or less). Thus, systemic use of HBOCs for prolonged tissue oxygenation, such as with chronic wounds, is contraindicated. Also, as previously described, systemic delivery of oxygen to dermal wound tissue relies on vascular sufficiency at the wound site. Thus, ischemic wounds and wounds otherwise complicated by vascular insufficiency are not good candidates for receiving oxygen delivered systemically via HBOCs. Furthermore, systemic use of HBOCs has been characterized by problems with unpredicable toxicities, such as severe vasoconstriction complications, arising from circulatory interaction with a variety of tissues, blood components, and biological pathways in the host.

Hypoxia is also a characteristic of harvested organ tissues (for example, skin, heart, kidney, liver, and eyes). Because hypoxia makes the organ tissue susceptible to oxidative stress, numerous preservation solutions have been developed to reduce hypoxia in harvested tissues so that the tissues can be maintained until implantation. The harvested tissues are immersed in such solutions until transplantation. Thus, if the solution is comprised of HBOCs, the unpredicable toxicities seen with systemic use of HBOCs may be of concern once the tissue is implanted.

The present invention relates to novel methods of using HBOCs to topically deliver oxygen to hypoxic tissues, thereby facilitating wound healing and/or reducing oxidative stress in harvested transplant tissue.

### SUMMARY OF THE INVENTION

The present invention provides methods, delivery devices, and compositions for topically oxygenating hypoxic mammalian tissue.

In various embodiments, the invention provides methods of treating mammalian hypoxic dermal tissue at a wound site, wherein a hemoglobin-based oxygen carrier (HBOC) is topically applied to the wound. In certain embodiments, the HBOC is Oxyglobin (Oxyb).

In certain embodiments, the invention provides methods of increasing the rate of dermal wound healing in mammals, wherein HBOC is topically applied to a wound in an amount that is about two to twelve mg HBOC/cm² wound. In certain embodiments, the amount applied is from about seven to eleven mg HBOC/cm² wound. In certain embodiments, the amount applied is from about ten to eleven mg HBOC/cm² wound.

In certain embodiments, the invention provides a delivery device, comprising HBOC and a carrier material, for administration of HBOC to mammalian wound tissue wherein the device releases HBOC at a sustained rate over a period of time.

In certain embodiments, the invention provides a composition, comprising HBOC and a pharmaceutically acceptable carrier, for treating mammalian wound tissue wherein a unit dose of the composition comprises from about two to twelve HBOC/cm² wound. In certain embodiments, the composition delivers from about seven to eleven mg HBOC/cm² wound. In certain embodiments, the composition delivers from about ten to eleven mg HBOC/cm² wound.

In certain embodiments, the invention can be used to treat acute and chronic dermal wounds. Such wounds can be chosen from full-thickness wounds and partial-thickness wounds. In certain embodiments, the wounds are ischemic wounds.

In certain embodiments, the invention can be used to treat wounds in a clinical setting and/or an extra-clinical setting. In certain embodiments, the extra-clinical setting can be chosen from a home, trauma setting, or field of combat.

In various embodiments, the invention also provides methods of treating mammalian post-harvest transplant tissue with a hemoglobin-based oxygen carrier (HBOC) that is topically applied to the tissue. In certain embodiments, the HBOC is Oxyglobin (Oxyb).

In certain embodiments, the HBOC is topically applied to harvested tissue in an amount that is about two to twelve mg HBOC/cm² tissue. In certain embodiments, the amount applied is from about seven to eleven mg HBOC/cm² tissue. In certain embodiments, the amount applied is from about ten to eleven mg HBOC/cm² tissue.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of the specification, illustrate embodiments of the invention and together with the description, serve to explain the principles of the invention.

FIG. 1 shows a summary overview of tissue oxygenation, factors involved in manipulation of tissue oxygenation and mechanisms and outcomes of tissue oxygenation. The triangular scale represents wound pO₂. Hypoxia, acutely induces angiogenic factors but relies on O₂ presence for functional regeneration of vessels. Sustained hypoxia (as in problem wounds) shuts down the HIF path via aHIF. Two approaches of oxygenation (HBOC & O₂, arrows) may be implemented to elevate tissue pO₂ in pigs. Restoration of O₂ in wounds (examined by oximetry) supports contraction, closure and angiogenesis. Correction of wound hypoxia will drive O₂-sensitive processes.

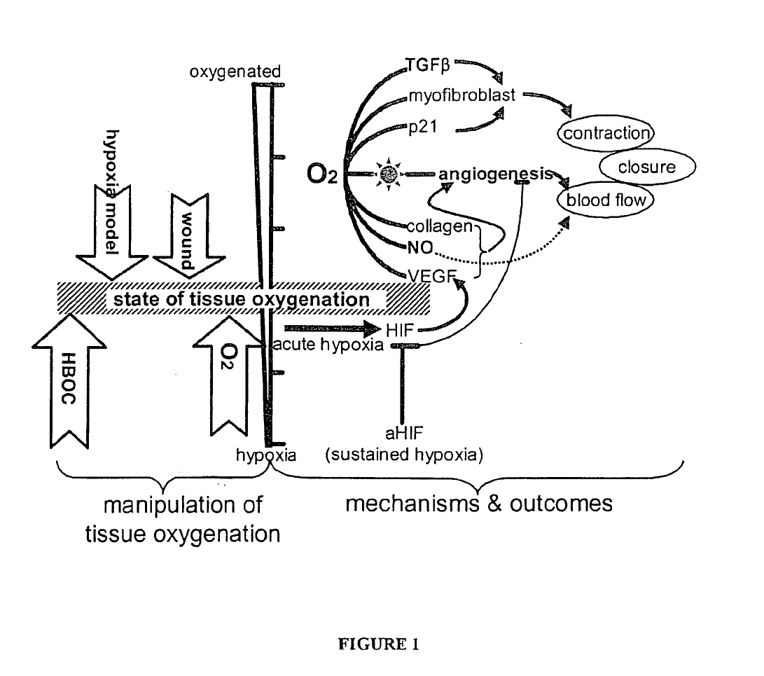

FIG. 2 shows wound-edge pO₂ in mice under hypoxic and normoxic conditions. After wounding mice were exposed to 10% O₂ (solid bar) or room air (open bar) for 24 h. pO₂ at wound-edge (1 mm margin) was determined using OxyLite (Oxford-Optronix)*, p<0.05.

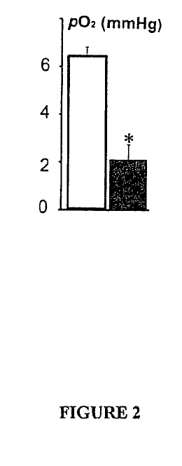

FIG. 3 shows that hypoxia in the wound-edge of hypoxic mice (see FIG. 2) impairs angiogenesis. Human dermal microvascular endothelial cells (HDMEC) were subjected to 24 h in Matrigel. Cells were labeled with calcein-AM. Bar graph, tubular length were blindly quantitated using Axiovision software (Zeiss). n=3. Mean±SD. *, p<0.05.

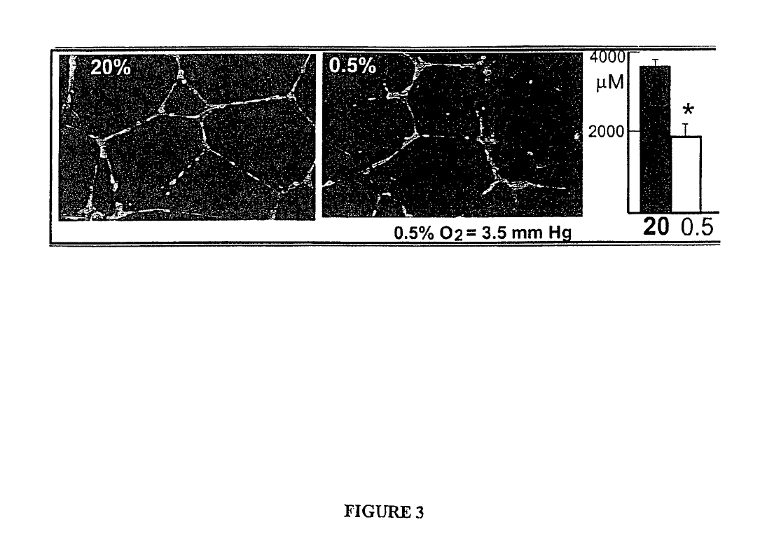

FIG. 4 shows that hypoxia potently delays wound closure. Two 8×16 mm full-thickness excisional wounds (inset) were placed on the dorsal skin of 8 wk old C57BL/6 male mice. Mice (n=5) were maintained in the following conditions: A. Post-wounding, mice were either held in 10% O₂ (hypoxia) or room air ambience (room air) from days 0-14. *, p<0.05 versus room air; B. On days (−1) before wounding, mice were held in 10% O₂ (pre-hypoxia) or room-air followed by holding them in 10% O₂ from days 0-14. Pre-hypoxia, held in 10% O₂ since day-1; hypoxia, held in 10% O₂ since day 0. *, p<0.05 versus hypoxia; C. Mice were maintained in room air ambience for the first 3 days (day 0-2) after wounding followed by holding them in 10% O₂ from days 3-14 (late hypoxia). Hypoxia, as in FIG. 4B. *, p<0.05 versus late-hypoxia. Wound area are shown as % area of initial wound size. Data shown as mean±SD.

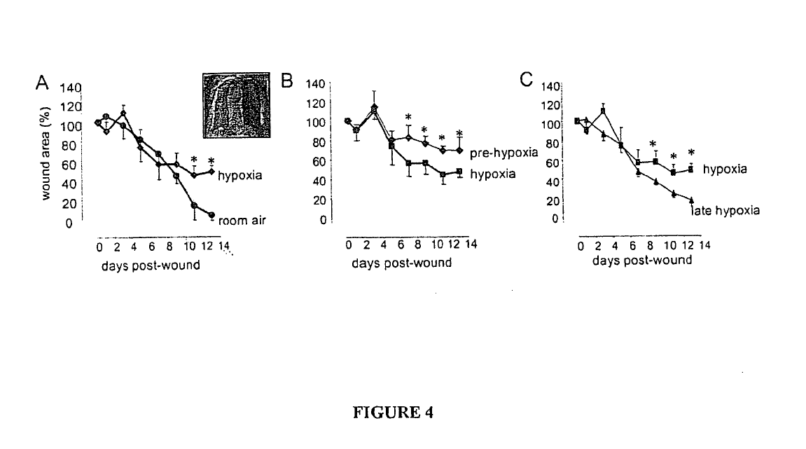

FIG. 5 shows that ex vivo oxygenation of hypoxic HBOC (HHBOC) and application of the resulting oxygenated HBOC (OHBOC) to hypoxic wounds accelerates wound closure. A. Oxygenation of Oxyglobin. Oxyglobin (Oxyb) is shipped under anoxic conditions to lengthen shelf life. pO₂ of the shipped hypoxic Oxyb (HOxyb) was detected (OxyLite) as 3-5 mm Hg. Oxygenated Ob (OOxyb) was generated by bubbling HOxyb with pure O₂ for 5 minutes followed by exposing the Oxyb to 100% O₂ at 4 ATM pressure (HBO) for 2 h. Following such exposure, the chamber was decompressed and the sample taken out for pO₂ assay. Because the pO₂ of the sample was read off-scale (beyond upper limit) in the OxyLite system, electrochemical probes (Apollo 4000, WPI) were used as alternative approach. Following HBO, peak pO₂ in OOxyb was close to 400 mm Hh. High pO₂ in OOxyb was maintained over time such that after 1 h (20 min lag time+40 min reading) following HBO exposure, pO₂ of OOxyb held over 250 mm Hg. In Hb-free buffer, pO₂ increases under HBO conditions but the pO₂ sharply declines and returns to baseline within 5 minutes after HBO exposure (not shown). B & C. Paired wounds (FIG. 5; n=4, 8 wk, males) were studied. All mice were maintained in hypoxia (10% O₂) since wounding. In each mouse, one of the two paired wounds were treated with OxyOb while the other wound was treated with HOxyb. Following 5 h after the first treatment, wound-edge PO₂ was recorded (B). Application of OOxyb significantly elevated wound-edge pO₂ (OxyLite) compared to the paired reading from the wound treated with HOxyb. The difference was highly significant even after 5 h of treatment indicating effective long-term oxygenation of the wound tissue in response to topical OOxyb application (B). C. Repeated treatment of the paired wounds, twice a day for the first four days of wounding, resulted in significant acceleration of wound closure. *, p<0.05, significantly different compared to pair-matched HOxyb treated control wound. n=5. Data are mean±SD. HBOC=HB-based O₂ carrier.

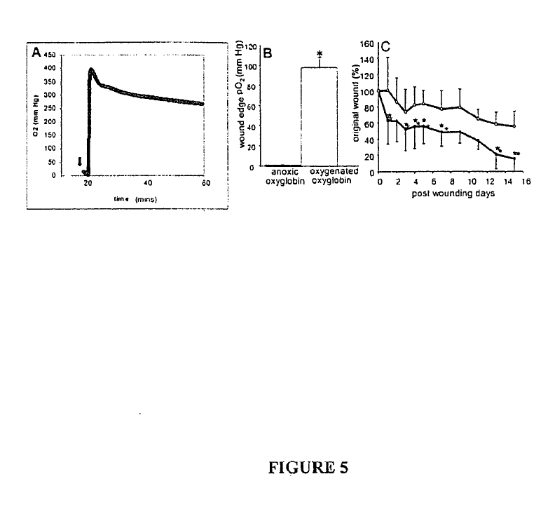

FIG. 6 shows that oxygenation of full-thickness dermal wounds promotes the rate of wound closure in swine. Ten (two clusters of five in the thoracic and lumbar areas of the back) secondary-intention full-thickness excisional dermal wounds (1×1 inch) were inflicted in pigs. Digital images of wounds on days 0 and 23 after wounding are shown in the inset. Five of ten wounds in each pig were treated with pure oxygen (open circles) for 3 h using a topical oxygen treatment device at a flow rate of 3-6/min. This treatment was performed every day for the first seven days (day 0-6) from the day of wounding. Five of the control wounds (solid circles) were exposed to room air for the similar period.

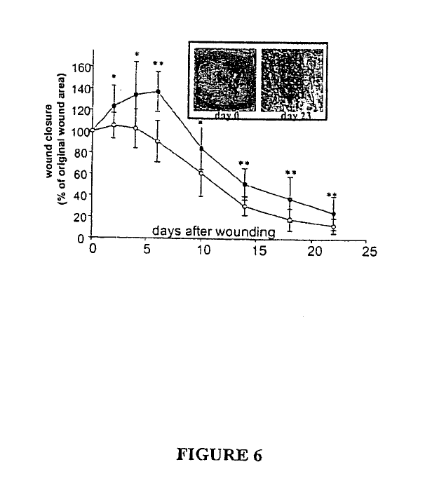

FIG. 7 shows that oxygenation of hypoxic oxyglobin (HOxyb) and topical application of the resulting oxygenated Oxyglobin (OOxyb) sustainably increases wound-bed pO₂ in swine. pO₂ measurement was performed non-invasively using Oxy-Lite (Oxford-Optronix). An O₂ electrode was specially designed for our application (pO₂ assay at 2 mm depth) by the vendor. A, real time measurement of pO₂; arrow indicates the time of initiation of local O₂ treatment. B, pO₂ levels in control (room air) and treated wounds on day 22. Treatment increased angiogenesis resulting in higher basal pO₂. n=3; Mean±SD. *, p<0.05.

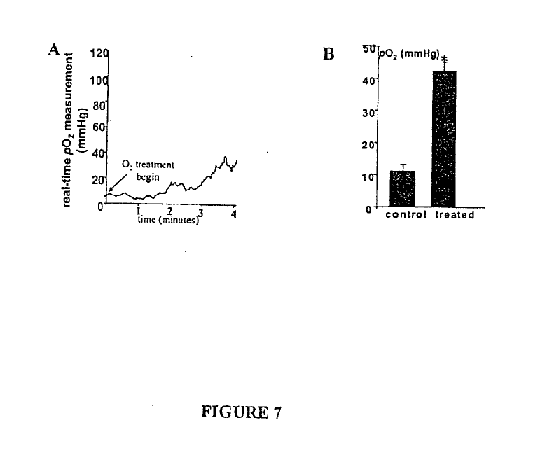

### DETAILED DESCRIPTION OF THE EMBODIMENTS

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such numerical ranges were all expressly written herein.

The present invention relates generally to methods, delivery devices, and compositions for topically oxygenating hypoxic tissue in order to promote wound healing and/or reduce oxidative stress in post-harvest transplant tissue. In some embodiments, the rate of tissue regeneration is increased, and in some embodiments, there is a reduction in scar tissue. The invention can generally be used to treat any damage to a living body in which the body's natural repair process will occur. The invention can be used to treat hypoxic tissue from animals, such as mammals, and specifically including humans.

In certain embodiments, the invention can be used to treat wounds in a clinical setting and/or an extra-clinical setting. In certain embodiments, the extra-clinical setting can be chosen from a home, a trauma setting (such as an accident scene), or field of combat.

In certain embodiments, the invention provides methods of treating mammalian hypoxic dermal tissue at a wound site, wherein a hemoglobin-based oxygen carrier (HBOC) is topically applied to the wound. In certain embodiments, the HBOC is a stabilized hemoglobin product of animal or artificial/recombinant origin, such as Oxyglobin® (Biopure Corp., Cambridge, Mass., USA); Hemopure® (Biopure Corp., Cambridge, Mass., USA); Hemolink™ (Hemosol, Inc., Toronto, Ontario, Canada); Hemospan® (Sangart, Inc., San Diego, Calif., USA); PolyHeme® (Northfield Laboratories, Inc., Evanston, Ill., USA); and OxyVita™ (Oxyvita, Inc., New Windsor, N.Y., USA). In certain embodiments, the HBOC is Oxyglobin (Oxyb). Because the HBOCs of the present invention are applied ex vivo to wound tissue, only localized interaction with tissue, blood components, and biological pathways occurs and thus, toxicity and the other problems characterizing systemic HBOC use are minimal.

In certain embodiments, the invention provides methods of treating mammalian hypoxic post-harvest transplant tissue in order to prevent oxidative stress, wherein a hemoglobin-based oxygen carrier (HBOC) is topically applied to the tissue. In certain embodiments, the HBOC is a stabilized hemoglobin product of animal or artificial/recombinant origin, such as Oxyglobin® (Biopure Corp., Cambridge, Mass., USA); Hemopure® (Biopure Corp., Cambridge, Mass., USA); Hemolink™ (Hemosol, Inc., Toronto, Ontario, Canada); Hemospan® (Sangart, Inc., San Diego, Calif., USA); PolyHeme® (Northfield Laboratories, Inc., Evanston, Ill., USA); and OxyVita™ (Oxyvita, Inc., New Windsor, N.Y., USA). In certain embodiments, the HBOC is Oxyglobin (Oxyb). Because the HBOCs of the present invention are applied topically to transplant tissue, only localized interaction with the tissue occurs. Thus, when the tissue is implanted, toxicity and the other problems characterizing systemic presence of HBOCs are of minimal concern.

The term “HBOC” is used herein in its generic sense, meaning that it encompasses both anoxic (HHBOC) and oxygenated (OHBOC) forms of a hemoglobin-based oxygen carrier. Unless the context of usage specifically dictates otherwise, no distinction is intended between the terms “HBOC,” “HHBOC,” and “OHBOC.” Accordingly, in some embodiments wherein the HBOC is Oxyglobin, the terms “Oxyglobin” and “Oxyb” are used herein in the generic sense, meaning that they encompass both anoxic Oxyglobin (HOxyb) and oxygenated Oxyglobin (OOxyb). Unless the context of usage specifically dictates otherwise, no distinction is intended between the terms “Oxyglobin,” “Oxyb,” “HOxyb” and “OOxyb.”

The term “wound” is used herein in its generic sense, meaning that it encompasses a variety of wounds, lesions, and injuries. Wounds can be full thickness (i.e., penetrating all layers of skin) or partial thickness (i.e. penetrating less than all layers of skin). Wounds can be acute or chronic, and acute wounds can become chronic over time. Examples of wounds include, but are not limited to, surgical wounds, penetrating wounds, avulsion injuries, crushing injuries, shearing injuries, burn injuries, lacerations, bite wounds, and ulcers (such as arterial ulcers, venous ulcers, pressure ulcers, and diabetic ulcers).

In certain embodiments, the invention is directed to treating and promoting the healing of ischemic tissues. Examples of ischemic tissues include, but are not limited to, post-operative wounds, wounds arising from peripheral vasculopathy (for example, diabetes, arteriosclerosis), wounds arising from arterial hypoxia (for example, pulmonary fibrosis or pneumonia, sympathetic pain response, hypothermia, anemia caused by major blood loss, cyanotic heart disease, high altitude); and post-harvest tissue for use in transplantation (for example, skin, heart, lung, eye, kidney, and liver).

In certain embodiments, the invention is directed to treating and promoting the healing of wounds that are on locations of the body that are impractical for topical administration of gaseous oxygen and/or are where minimal scarring is desired (for example, with facial wounds).

In certain embodiments, the invention is directed to treating and promoting the healing of hypoxic wounds wherein the hypoxia is related to wound infection, or other causes.

In certain embodiments, the invention provides methods of oxygenating HHBOC ex vivo. For example, HOxyb (pO₂ about 3-5 mmHg) is bubbled with pure oxygen for 5 minutes, followed by exposure (in a hyperbaric chamber) to 100% oxygen at 4 ATM pressure for 2 hours, resulting in highly saturated OOxyb (pO₂ about 250-400 mmHg) that can maintain high saturation levels over time. In certain embodiments, the steps of oxygenating HHBOC can be performed in a single device that connects to an oxygen source and that can maintain a pressure of about 2-4 ATM. Such device can be disposable or reusable, and can be configured for use in a clinical or extra-clinical setting.

In certain embodiments, the invention provides methods of topically delivering OHBOC to hypoxic wound tissue in a manner such that oxygen can be gradually released from OHBOC directly to wound tissue. In certain embodiments, topical application of an OHBOC stimulates angiogenesis and blood flow, reduces wound dilation, and supports wound contraction.

In certain embodiments, the invention provides methods of topically delivering OHBOC to harvested organ tissue, which is characterized by hypoxia, in a manner such that oxygen can be gradually released from OHBOC directly to the tissue. In certain embodiments, topical application of an OHBOC stimulates the proliferation of granulation tissue, stimulates vessel growth, and promotes epithelial healing.

In certain embodiments, the invention provides methods of increasing the rate of wound healing in animals comprising applying OHBOC to a wound in a quantity sufficient to raise the level of oxygen in the wound tissue from hypoxic levels to sustained normoxic levels. For example, in humans OHBOC could be applied in sufficient quantity to raise pO₂ to at or above 50-60 mmHg. In certain embodiments, OHBOC is applied in a quantity of about two, three, four, five, six, seven, eight, nine, ten, eleven, or twelve mg OHBOC per square centimeter of wound. In certain embodiments, OHBOC is applied in a quantity of about seven, eight, nine, ten, or eleven mg OHBOC per square centimeter of wound. In certain embodiments, OHBOC is applied in a quantity of about ten OHBOC per square centimeter of wound. Thus, for example, Oxyb can be applied in a quantity of about 10.3 mg Oxyb per square centimeter of wound.

In certain embodiments, the invention provides methods of preventing/reducing hypoxia in post-harvest transplant tissue comprising topically applying OHBOC to the tissue. For example, for human organ tissue, OHBOC could be applied in sufficient quantity to raise pO₂ to 100 mmHg. In certain embodiments, OHBOC is applied in a quantity of about five, six, seven, eight, nine, ten, eleven, twelve, thirteen, fourteen, fifteen, sixteen, seventeen, eighteen mg OHBOC per square centimeter of tissue. In certain embodiments, OHBOC is applied in a quantity of about seven, eight, nine, ten, eleven, twelve, thirteen, fourteen, or fifteen mg OHBOC per square centimeter of tissue. In certain embodiments, OHBOC is applied in a quantity of about ten OHBOC per square centimeter of tissue. Thus, for example, Oxyb can be applied in a quantity of about 10.3 mg Oxyb per square centimeter of tissue.

In certain embodiments, a dosage of HBOC is applied to a wound for a period of time. The period of time can be one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours, or more. For example, a dosage of Oxyb can be applied to a wound for 4-6 hours.

In certain embodiments, HBOC is topically applied to post-harvest transplant tissue for a period of time. The period of time can vary as tissue transportation needs dictate but can nevertheless be one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours, or more.

In certain embodiments, an HBOC is applied to a wound during the early (inflammatory) and late (tissue remodeling) phases of wound healing. In certain embodiments, the HBOC is applied to a wound during the early phase of wound healing in order to reduce wound dilation and induce contraction.

In certain embodiments, the invention provides a delivery device, comprising a HBOC, (for example, Oxyb) and a carrier material, for administration of the HBOC to mammalian hypoxic tissue wherein the device releases the HBOC in an active form at a sustained rate for a period of time. In certain embodiments, the period of time can be one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours, or more. In certain embodiments, the delivery device allows oxygen to diffuse to the hypoxic tissue.

Delivery devices include, but are not limited to, bandages, surgical dressings, gauzes, adhesive strips, surgical staples, clips, hemostats, and sutures. Devices according to the invention can be prepared according to known methods, and can include, or be made from, polymeric material. In some instances, the polymeric material will be an absorbable material and in other instances, a non-absorbable material. Devices can, of course, include both absorbable and non-absorbable materials.

Absorbable materials can be synthetic materials and non-synthetic materials. Absorbable synthetic materials include, but are not limited to, cellulosic polymers, glycolic acid polymers, methacrylate polymers, ethylene vinyl acetate polymers, ethylene vinyl alcohol copolymers, polycaptrolactam, polyacetate, copolymers of lactide and glycolide, polydioxanone, polyglactin, poliglecaprone, polyglyconate, polygluconate, and combinations thereof. Absorbable non-synthetic materials include, but are not limited to, catgut, cargile membrane, fascia lata, gelatin, collagen, and combinations thereof.

Nonabsorbable synthetic materials include, but are not limited to nylons, rayons, polyesters, polyolefins, and combinations thereof. Non-absorbable non-synthetic materials include, but are not limited to, silk, dermal silk, cotton, linen, and combinations thereof.

In certain embodiments, the invention provides a composition, comprising a HBOC (for example, Oxyb) and a pharmaceutically acceptable carrier, for application to mammalian hypoxic tissue wherein a unit dose of the composition comprises a HBOC. In certain embodiments, the composition delivers from about two to twelve mg HBOC/cm² wound. In certain embodiments, the composition delivers from about seven to eleven mg HBOC/cm² wound. In certain embodiments, the composition delivers from about ten to eleven mg HBOC/cm² wound. In certain embodiments, the composition allows oxygen to diffuse to the wound tissue. In certain embodiments, the composition delivers from about five to eighteen mg HBOC/cm² transplant tissue. In certain embodiments, the composition delivers from about seven to fifteen mg HBOC/cm² transplant tissue. In certain embodiments, the composition delivers from about ten to eleven mg HBOC/cm² transplant tissue.

In a composition, the HBOC can be applied in any form of pharmaceutically acceptable carrier, including but not limited to, liquids, gels, lotions, creams, pastes, and ointments. The means of application will depend upon what form the HBOC takes. For example, liquids can be sprayed or poured, and gels, lotions, creams, pastes, and ointments can be rubbed or massaged.

A composition can be homogenous (for example, forms in which the HBOC is in solution) or heterogeneous (for example, forms in which the HBOC is contained within liposomes or microspheres). The composition can produce an immediate effect, and can alternatively, or additionally, produce an extended effect. For example, liposomes, or microspheres, or other similar means of providing an extended release of the HBOC, can be used to extend the period during which the HBOC is exposed to the hypoxic tissue. Non-encapsulated HBOC can also be provided for an immediate effect.

Combinations of the aforementioned delivery devices and compositions are also envisioned. For example, an HBOC-containing gel or ointment can be impregnated into a bandage or wound dressing for delivery of the HBOC to the desired location. As another example, an absorbable device can be loaded with an HBOC solution and release the solution from the device over a period as desired. The physical form used to deliver the HBOC is not critical and the choice or design of such devices is well within the level of skill of one in the art.

The HBOC is delivered to the desired target site at least once. In some embodiments, the frequency of delivery can be as often as every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours, or more. In some embodiments, the delivery time can be over a period of days or weeks, and at intervals of one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours over one, two, three four, five or six weeks or more. In some embodiments, the delivery time can be for as long as every one, two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four hours, or more. Where repeated doses are desired, devices or other carriers can be “programmed” to release doses of the HBOC at desired times. For example, a microsphere formulation can include unencapsulated HBOC for an immediate effect on administration, an encapsulated component to deliver a second dose at twenty-four hours, and an encapsulated component to deliver a third dose at forty-eight hours. The treatment strategy is left to the practitioner, and the design of devices, compositions, formulations, and carriers is within the level of skill in the art.

It may be desirable to provide for other conditions in the practice of the present invention. For example, it may be desirable to maintain a desired level of moisture and a particular temperature; in some embodiments, a warm, moist environment is desirable. While not required, it may also be desirable to establish or maintain a sterile environment.

Additionally, it may be desirable to include other therapeutically beneficial agents in the formulation. For example, the vehicles or carriers may also include humectants or moisturizers to maintain a desired moisture level in the treated area. Other possibilities include drugs such as anesthetics or antibiotics, which provide other desired effects. Again, the possibilities are unlimited and are left to the practitioner.

The following Examples are provided to even more clearly describe and explain the invention.

### EXAMPLES

### Example 1

### Study of Oxyb-Facilitated Wound Healing in Pigs

Before Oxyb is used for treating a wound, it is oxygenated by bubbling hypoxic Oxyb (HOxyb; pO₂<5 mm) with pure oxygen for 5 minutes, followed by exposure to 100% oxygen (at 4 ATA) for 2 hours.

Pigs are sedated using Telazol (tiletamine and zolazepam, 6 mg/Kg). During wounding and treatment, animals are kept anesthetized with isoflurane via a face cone. The wound sites over the dorsal trunk area are shaved using a #40 clipper blade and the area cleaned with alcohol and Betadine solution. Ten dermal wounds (5 thoracic and 5 lumbar) are created using a #15 scalpel by removing a full-thickness 6.3 cm² section of skin. Of the ten wounds, every alternate wound is topically treated with 0.5 mL of OOxyb prepared as previously described. The control wounds are treated with HOxyb. Wounds are treated for once daily for the first seven days after wounding. The daily treatment involves topical application of the Oxyb for three hours, followed by a new wound dressing of moist Tegaderm pads held in place with Elasticon tape. After the seven-day treatment period is completed, wound dressings are changed every fourth day (when biopsy is also collected) until the wounds are completely closed and the scab falls off.

Analysis of whether topical treatment of wound tissue with OOxyb corrects wound hypoxia can be performed using wound oximetry to measure wound surface pO₂. Wound healing can be assessed by evaluating the rates of wound re-epithelialization and closure. The open wound area and the area enclosed by the normal hair bearing skin can be measured using a macrophotographic technique. The healing rate can be monitored every day for the first seven days. After this period, the wounds can be imaged and biopsy collected from designated wounds in each set) on every fourth day during change of dressing. Wound contraction, as a percentage of the original wound size, can be calculated by monitoring the wound area (WoundMatrix™). Data can be expressed as a percentage compared to area of wound at day zero.

Detailed evaluation of the regenerating tissue can be performed by measuring the following markers: (i) collagen content: assessed by determining the hydroxyproline content using Ehrlich's reagent; (ii) tensile strength: determined with a tensiometer as a function of breaking force per cross-sectional area of tissue; and (iii) histology: biopsis can be collected on days zero, three, seven, and every fourth day thereafter from the designated wound in each set. The biopsies can be fixed in formalin and stained with hematoxylin and eosin as well as Masson Trichrome to detect general wound-tissue architecture.

Other embodiments of the invention will be apparent to those skilled in the art from consideration of the specification and practice of the invention disclosed herein. It is intended that the specification and examples be considered as exemplary only, with true scope and spirit of the invention being indicated by the following claims.

What is claimed is:

1. A method of increasing the rate of wound healing in animals comprising applying to a wound an oxygenated hemoglobin-based oxygen carrier (“OHBOC”) in a quantity sufficient to raise the level of oxygen in the wound tissue to a level at or above 50-60 mmHg, wherein the OHBOC is administered topically to a wound at least once per day for a treatment period of at least two days.

2. The method according to claim 1, comprising applying about 2-12 mg of OHBOC per square centimeter of wound.

3. The method according to claim 2, comprising applying about 7-1 mg of OHBOC per square centimeter of wound.

4. The method according to claim 3, comprising applying about 10.3 mg of OHBOC per square centimeter of wound.

5. The method according to claim 1, wherein the treatment period is about 3-5 days.

6. The method according to claim 1, wherein the OHBOC is applied continuously to the wound over a period of from about 1 hours to about 24 hours.

7. The method according to claim 6, wherein the OHBOC is applied continuously to the wound over a period of from about 4 hours to about 6 hours.

8. The method according to claim 1, wherein the OHBOC is applied in the form of a pharmaceutically acceptable composition chosen from gels, lotions, ointments, creams, pastes, and liquids.

9. The method according to claim 1, wherein the OHBOC is applied in a pharmaceutically acceptable device chosen from bandages, surgical dressings, gauzes, patches, and adhesive strips.

10. The method according to claim 9, wherein the device comprises a polymeric material that is either a synthetic material chosen from cellulosic polymers, glycolic acid polymers, methacrylate polymers, ethylene vinyl acetate polymers, ethylene vinyl alcohol copolymers, polycaptrolactam, polyacetate, copolymers of lactide and glycolide, polydioxanone, polyglactin, poliglecaprone, polyglyconate, polygluconate, nylons, rayons, polyesters, polyolefins and combinations thereof, or a non-synthetic material chosen from catgut, cargile membrane, fascia lata, gelatin, collagen, silk, dermal silk, cotton, linen, and combinations thereof.

11. The method according to claim 1, wherein the wound is an acute or chronic surgical wound, penetrating wound, avulsion injury, crushing injury, shearing injury, burn injury, laceration, bite wound, arterial ulcer, venous ulcer, pressure ulcer, or diabetic ulcer.

12. A method of increasing the rate of wound healing in animals comprising;
providing a substrate comprising at least one hypoxic hemoglobin-based oxygen carrier (“HHBOC”) in a pharmaceutically acceptable composition;
subjecting the substrate to pressurization in a hyperbaric chamber under conditions suitable to render the HHBOC to an oxygenated form (“OHBOC”)
applying the substrate comprising the OHBOC to a wound for a period of time that is sufficient to maintain oxygenation in the wound tissue at or above 50-60 mmHg for a period of time of at least one hour.

13. The method according to claim 12, wherein oxygenation in the wound tissue is maintained at or above 50-60 mmHg for a period of time of at least one hour to six hours.

14. The method according to claim 12, wherein oxygenation in the wound tissue is maintained at or above 50-60 mmHg for a period of time of at least six hours to twenty-four hours.

15. An oxygen delivery device for administration of oxygen to a wound, comprising at least one HBOC in a hypoxic form (“HHBOC”) and a carrier material, wherein the device is configured to be exposed to pressurization in a hyperbaric chamber under conditions suitable to render the HHBOC to an oxygenated form (“OHBOC”), such that the delivery device, when applied to a wound on an animal, releases oxygen from the OHBOC in a quantity sufficient to raise the level of oxygen in the wound tissue to a level at or above 50-60 mmHg for a period of time from one hour to twenty four hours.

16. A method of reducing hypoxia in post-harvest mammalian donor organ tissue comprising applying to such organ tissue an oxygenated hemoglobin-based oxygen carrier (“OHBOC”) in a quantity sufficient to maintain oxygen levels at or above 100 mmHg, wherein the OHBOC is administered topically to the tissue for a period of time prior to implantation in a host.

17. The method according to claim 16, comprising applying about 5-18 mg of OHBOC per square centimeter of tissue.

18. The method according to claim 17, comprising applying about 7-15 mg of OHBOC per square centimeter of tissue.

19. The method according to claim 18, comprising applying about 10.3 mg of OHBOC per square centimeter of tissue.

20. The method according to claim 16, wherein the OHBOC is applied continuously to the tissue over a period from about 1 hours to about 48 hours.
